(19) United States
(12) Patent Application Publication
(10) Pub. No.: US 2024/0150692 A1
(43) Pub. Date: May 9, 2024

(54) ORGAN CROSSTALK IN VITRO CHAMBER TO SIMULATE MULTI ORGAN TISSUES

(71) Applicant: Government of the United States as represented by the Secretary of the Air Force, Wright-Patterson AFB, OH (US)

(72) Inventors: Saber M. Hussain, Beavercreek, OH (US); Richard Agans, Miamisburg, OH (US); Mark T. Nelson, Oakwood, OH (US); Richard L. Salisbury, Lewisburg, OH (US)

(21) Appl. No.: 18/476,464

(22) Filed: Sep. 28, 2023

Related U.S. Application Data
(60) Provisional application No. 63/423,689, filed on Nov. 8, 2022.

(51) Int. Cl. C12M 3/00; C12M 1/00; C12M 1/12

(52) U.S. Cl. CPC C12M 21/08; C12M 23/06; C12M 23/34; C12M 23/38; C12M 23/50; C12M 23/58; C12M 25/02; C12M 25/14; C12M 29/14

(57) ABSTRACT

In accordance with various embodiments of the disclosed subject matter, a system, device and platform for culturing and maintaining tissue representative cellular models combined with active fluidics mimicking body circulation. In combination, multiple chamber devices enable modeling of multi-organ communication, representative of in vivo conditions.

### GOVERNMENT INTEREST

The invention described herein may be manufactured and used by or for the Government of the United States for all governmental purposes without the payment of any royalty.

### FIELD OF THE DISCLOSURE

The present invention generally relates to a platform configured to enable dynamic study of organ-related functions such as tissue growth and circulatory system functions.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Living organs are three-dimensional vascularized structures composed of two or more closely apposed tissues that function collectively and transport materials, cells and information across tissue-tissue interfaces in the presence of dynamic mechanical forces. While microfluidic mimicking of an organ via 2D and 3D devices (e.g., scaffolding) for cell culturing is known, there are no such devices suitable for use in assessing dynamic organ functions such as circulatory issues and the like.

While there have been advances in the field of in vitro modeling for tissues, the current state of the art is still lacking an ability to fully represent single organs/tissues let alone multiple tissue systems. As such, there is a need to develop models which allow increased cell densities, model circulatory dynamics within the body, and enable incorporation of multiple quantitative assays.

### SUMMARY OF THE INVENTION

Various deficiencies in the prior art are addressed below by a system, device and platform for culturing and maintaining tissue representative cellular models combined with active fluidics mimicking body circulation. In combination, multiple chamber devices enable modeling of multi-organ communication, representative of in vivo conditions.

Various embodiments provide a platform configured to link multiple organ tissues via a singular vascular fluidics network(s) such as for in vitro studies of dynamic organ function including organ tissue growth and circulatory system functions. Various embodiments of the platform comprise a plurality of chambers with organ tissues and a tissue membrane/interface separation therebetween, further including at least one fluidics circuit connected to the chambers such that multiple organ tissues may be simulated simultaneously and interactions therebetween may be observed.

An apparatus according to an embodiment comprises a base, having a top surface and a bottom surface and having formed therein a plurality of open-top chambers and at least a first fluidics circuit; each open top chamber being divided into respective upper and lower sub-chambers by respective chamber separating membrane, wherein the upper and lower sub-chambers are defined by interior surfaces configured to allow organ culture substrates to be secured thereto; the first fluidics circuit configured to support a first fluid flow between a first port on the base and second port on the base, the fluidics circuit being further configured to support a fluid flow through a lower sub-chamber of at least one of the plurality of open top chambers.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIGS. 1-2 depict three-dimensional (3D) views of, respectively, a base portion and a lid portion of a platform according to an embodiment.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Various embodiments provide a system, device and platform for culturing and maintaining tissue representative cellular models combined with active fluidics mimicking body circulation. In combination, multiple chamber devices enable modeling of multi-organ communication, representative of in vivo conditions.

Various embodiments relate to a platform configured to link multiple organ tissues via one or more singular vascular fluidics networks such as for in vitro studies of dynamic organ function including organ tissue growth and circulatory system functions. Embodiments of the platform generally comprise a plurality of chambers with organ tissues and a tissue membrane/interface separation therebetween, further including one or more microfluidics or millifluidics circuits connected to the chambers such that multiple organ tissues may be simulated simultaneously and interactions therebetween may be observed. For example, the various embodiments may be used to simulate toxicological insult via respiratory, dermal, or oral exposure routes. The various embodiments have broader application potential for any studies where circulation drives responses in multiple tissues (i.e. infection, metabolism, transport, etc.).

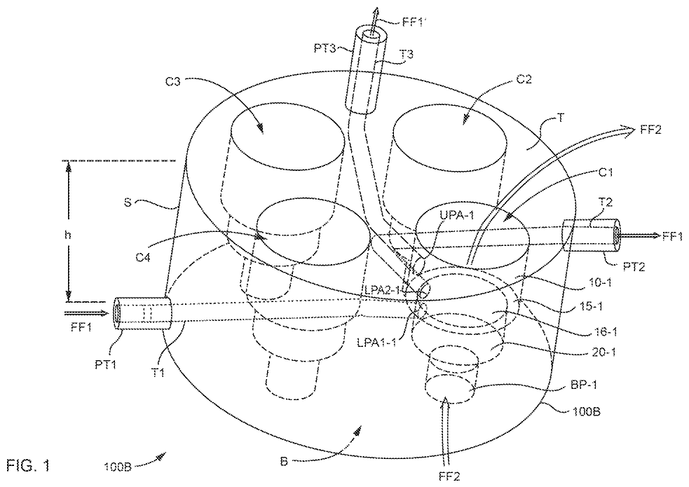

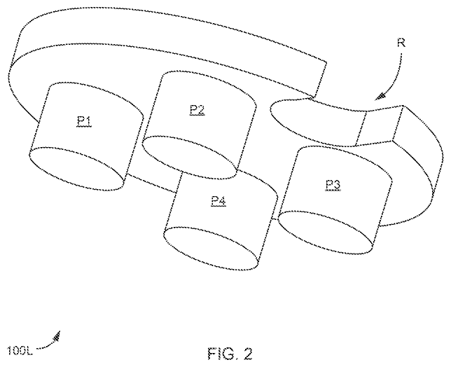

FIGS. 1-2 depict three-dimensional (3D) views of, respectively, a base portion 100B and a lid portion 100L of a platform 100 according to an embodiment. FIGS. 1-2 will be described together. It is noted that the base 100B and lid 100L portions of the platform 100 are depicted as being substantially cylindrical and forming thereby a substantially cylindrical platform 100 when coupled together. However, it will be appreciated that the platform 100, by its base 100B and/or lid 100B portions, may be formed in accordance with any shape suitable for use in performing the various functions described herein (e.g., cube, rectilinear, ovoid etc.). As such, the specific shape of the platform 100 depicted in the various figures is simply one embodiment of the platform 100 contemplated by the inventors.

In various embodiments, the base 100B and lid 100L portions of the platform 100 are manufactured using 3D printing techniques.

Referring to FIG. 1, the base portion 100B of the platform 100 is depicted as a substantially cylindrical body having a top surface T separated from a bottom surface B by a side S having a height h. Formed within the body are a plurality of chambers (illustratively four, denoted as chambers C1-C4). Each of the four chambers C1-C4 has a respective top surface opening (i.e., C1-C4 are open top chambers) and a respective bottom surface opening, where the size of the top surface openings are larger than the size of the respective bottom surface openings.

To simplify the discussion, and since each of the chambers C1-C4 are structurally similar, the first chamber C1 will be the primary focus of the following discussion. The structure/function discussed below with respect to first chamber C1 is generally applicable to each of the second C2, third C3 and fourth C4 chambers.

First chamber C1, which is formed within the body of the base portion 100B, comprises an elongated chamber having upper, center, and lower portions axially aligned therein, wherein a cross-sectional area normal to an axis therethrough exhibits a larger area at the upper portion and a smaller area at the lower portion (e.g., a cylindrically formed chamber having a decreasing diameter at its center portion compared to the upper portion). The chamber is defined by interior chamber surfaces or walls configured to allow organ culture substrates to be secured thereto. Such interior growth surfaces may comprise the native 3D printing material alone or treated to increase cell adhesion and growth, e.g., such as the bottom surface of sub-chamber 20-1. In other embodiments, sub-chambers 10-1 and 20-1 could each be filled with a growth matrix to allow modeling of internal organ structures in more complex dynamic studies. These matrices may be constructed of polystyrene, polycarbonate, or glass coated in known biopolymers (e.g., Matrigel). Alternatively, polydimethylsiloxane may be utilized as a growth substrate given its ability to be tuned to different stiffnesses for allowing the modeling of multiple tissue matrix types, and coated in collagen to promote cell adherence.

As depicted in FIG. 1, the first chamber C1 comprises an upper sub-chamber 10-1 and a lower sub-chamber 20-1 formed as axially aligned, cylindrical sub-chambers of differing annular diameters. The annular diameter of the upper sub-chamber 10-1 is greater than the annular diameter of the lower sub-chamber 20-1. An annular ring or ridge 15-1 at the lower portion or base of the upper sub-chamber 10-1 provides a substantially flat surface (normal to the axis of the chambers) which is used as the surface upon which a chamber separating membrane 16-1 is secured. The separating membrane 16-1 is configured to divide or separate the upper sub-chamber 10-1 and lower sub-chamber 20-1, and to provide that some or all of target cells harbored in the respective sub-chambers 10-1/20-1 may be isolated from each other by the sub-chamber separating membrane 16-1. It should be noted that in some embodiments where cells are grown on both sides of a separating membrane 16, the bottom surface of the corresponding sub-chamber 20 may be left unmodified and only serve as a chamber for fluidics movement purposes.

Also formed within the body of the base portion 100B are, for each of chambers C1-C4, two unique micro- or milli-fluidics circuits which allow respective separate flow dynamics into the two respective sub-chambers of each chamber. The first fluidics circuits allow first flows to be directed across the lower sub-chamber; entering through a first tube towards the bottom of the base and exiting through a second tube towards the top of the base. The second fluidics circuits allow second flows to enter from the bottom of the base and exit at the top of the base, such that multiple bases can be ‘linked’ together (i.e., in fluid communication with each other) for increased model complexity. Furthermore, in various embodiments the respective first and second integrated fluidics circuits are configured to provide fluid flows in a manner directed towards an incline such that the risk of bubble formation within the fluid flows is greatly reduced.

First Fluidics Flow(s)

Referring to FIG. 1 and in particular to first chamber C1, it can be seen that a first fluid flow FF1 is depicted as entering the base portion 100B via a port PT1 (illustratively depicted in FIG. 1 as being located at the lower left side of the base) associated with a first tube T1 formed within the base portion 100B. The first tube T1 conveys the first fluid flow F1 to a first lower portion aperture LPA1-1 in the side wall of the lower sub-chamber 20-1 (i.e., towards the bottom of the base) of first chamber C1. The first fluid flow F1 passes through/across the lower sub-chamber 20-1 (and any material therein), exiting the lower sub-chamber 20-1 via a second lower portion aperture LPA2-1 in the side wall of the lower sub-chamber 20-1, which is illustratively depicted as being above (i.e., closer to the top surface) the first lower portion aperture LPA1-1. A second tube T1 formed within the base portion 100B conveys the first fluid flow FF1 from the second lower portion aperture LPA2-1 to a port PT2 (illustratively depicted in FIG. 1 as being located at the upper right side of the base) associated with the base portion 100B.

It will be appreciated that while the first and second lower portion apertures LPA1-1/LPA2-1 are shown in FIG. 1 and described herein as stacked (i.e., one above the other), in various embodiments these apertures may be provided in differing configurations, such as being located next to each other, or on opposite sides of the lower sub-chamber 20-1, or some combination thereof. Further, while the first and second lower portion apertures LPA1-1/LPA2-1 and tubes T1/T2 are shown in FIG. 1 as being of substantially the same cross-sectional size, in various embodiments the apertures and/or respective portions of first or second tubes T1/T2 may be of differing sizes.

Optionally, a third tube T3 (illustratively depicted in FIG. 1 as being located at the top of the base) formed within the base portion 100B conveys a portion FF1′ of the first fluid flow FF1 capable of passing through the chamber separating membrane 16-1 from an upper portion aperture UPA-1 in the side wall of the upper sub-chamber 10-1 to a port PT3 associated with the base portion 100B.

It will be appreciated that while the upper portion aperture UPA-1 is depicted in a particular location in FIG. 1, in various embodiments this aperture may be provided in differing sizes and/or locations within the upper sub-chamber 10-1. Further, while the upper portion aperture UPA-1 and third tube T3 are shown in FIG. 1 as being of substantially the same cross-sectional size, in various embodiments the aperture and/or respective portions of third tube T3 may be of differing sizes.

As depicted in FIG. 1, the first tube T1 connects the first port PT1 to a first lower portion aperture LPA1 of the lower sub-chamber 20 of each of the chambers C1-C4. However, in various embodiments first tube T1 may be configured to connect less than all of the (illustratively four) chambers formed within a particular base portion 100B of a platform 100.

As depicted in FIG. 1, the second tube T2 connects the second port PT2 to a second lower portion aperture LPA2 of the lower sub-chamber 20 of each of the chambers C1-C4. However, in various embodiments second tube T2 may be configured to connect less than all of the (illustratively four) chambers formed within a particular base portion 100B of a platform 100.

As depicted in FIG. 1, the third tube T3 connects the third port PT3 to an upper portion aperture UPA of the upper sub-chamber 10 of each of the chambers C1-C4. However, in various embodiments third tube T3 may be configured to connect less than all of the (illustratively four) chambers formed within a particular base portion 100B of a platform 100.

The above-described microfluidic flow direction for first fluid flow FF1 is for illustrative purposes only. In various embodiments, the fluid flow between any of the ports is configured/selected in accordance with a desired use case such as replicating fluid flow of a particular organ, organelle, or other structure.

Second Fluidics Flow(s)

Referring to FIG. 1, and in particular to first chamber C1, it can be seen that a second fluid flow FF2 is depicted as entering the lower sub-chamber 20-1 via a bottom port BP-1 in the bottom surface B of base portion 100B. The second fluid flow FF2 is depicted as passing through/across the lower sub-chamber 20-1 (and any material therein), through the chamber separating membrane 16-1, through/across the upper sub-chamber 10-1, and exiting the upper sub-chamber 10-1 via a top port TP-1 in the top surface T of base portion 100B.

The above-described microfluidic flow direction for second fluid flow FF2 is for illustrative purposes only. In various embodiments, the fluid flow between any of the ports is configured/selected in accordance with a desired use case such as replicating fluid flow of a particular organ, organelle, or other structure.

In various embodiments, some or all of the (illustratively four) chambers C1-C4 are configured to receive and exit a respective second fluid flow FF2 as described above with respect to first chamber C1.

Combined Fluidics Flow(s)

In various embodiments, any of the structures such as the tubes, ports, chambers, chamber separating membranes and the like configured/selected in accordance with a desired use case, such as replicating fluid flow of a particular organ, organelle, or other structure.

Such selections may result in dynamic changes to the pressure, velocity, content and other parameters of the various microfluidic flows. For example, assuming that the first FF1 and second FF2 fluid flows through first chamber C1 are provided substantially as describe above, portions of the second fluid flow FF2 may exit the first chamber C1 via ports other than the top port TP, and portions of the first fluid flow FF1 may exit the first chamber C1 via ports other than side ports on the chamber C1.

Base and Lid Configurations

Referring to FIG. 2, the lid portion 100L of the platform 100 is depicted as being configured to mate to the top surface T of the base portion 100B of the platform 100. As depicted, the base portion 100B comprises, illustratively, four protrusions denoted as P1-P4 which are configured to mate with or be received by corresponding chambers C1-C4 and form thereby a tight seal therebetween. Further depicted is a recess R formed within the lid portion 100L and configured to allow protrusion therethrough of the third port PT3 described above with respect to the base portion 100B of the platform 100.

Further, the base portion 100B may include a plurality of recesses in its bottom surface which line up with a corresponding plurality of protrusions on the lid portion 100L as discussed herein, thus ensuring a secure mechanical connection and operational cooperation between two individual devices.

Single Chamber Configurations

Single chamber embodiments may be realized by extending all of the lower protrusions P1-P4 of the lid 100L such that, when the lid is mated to the base, the extended lower protrusions mechanically cooperate with respective culture substrate ridges 15 of the upper sub-chambers 10 (thereby sealing the upper sub-chambers 10-1 through 10-4 of the chambers C1-C4). In these embodiments, since the second and/or third fluidics circuits associated with the upper sub-chambers 20-1 through 20-4 are blocked or sealed by the insertion therein of the lid protrusions P1-P4, only the first internal fluidics circuit is used. In this manner cells may still be cultured in the lower sub-chamber 20 via secured membrane 16 or the floor of the lower sub-chamber 20.

Combined single and multiple chamber embodiments may be realized by extending some of the lower protrusions P1-P4 of the lid 100L such that, when the lid is mated to the base, the extended lower protrusions mechanically cooperate with respective culture substrate ridges 15 of respective upper sub-chambers 10 to thereby seal the respective upper sub-chamber 10 of corresponding chambers C. In these embodiments, only the first internal fluidics circuit is used for a chamber C where the upper sub-chambers 10 has been sealed. In this manner cells may still be cultured in the lower sub-chamber 20 via secured membrane 16 or the floor of the lower sub-chamber 20.

Stacked Platforms

Various embodiments contemplate that multiple base portions 100B of the platform may be “stacked” in that top surface ports (TP-1 through TP-X, where X is the number of chambers) are aligned with corresponding bottom surface ports (BP-1 through BP-X) such that, when stacked, the aligned ports are configured to support fluid communication therebetween.

For example, the second fluidics path contemplates flow entering from the bottom of the base and exiting the top of the base such that multiple bases can be ‘linked’ together for increased model complexity. Furthermore, the various fluidics circuits (apertures, tubes, paths etc.) may be configured to provide a microfluidic or millifluidic fluid flow that is always directed towards an incline such that the risk of bubble formation therein is greatly reduced.

General Use Cases

Various embodiments enable rapid assessment of circulatory issues associated with organ models constructed in accordance with the chambers provided in the disclosure, such as where the first milli/micro-fluidics circuit represents a singular vascular fluidics network for in vitro studies of organ tissues cultured within one or more chambers. Specifically, by representing vascular and interstitial fluid dynamics, connecting multiple devices via the vascular fluidics network enables modeling of multiple organ systems together and assessing the role of circulation during toxicological insults. The resulting device/platform may be used to simulate toxicological insult via respiratory, dermal, or oral exposure routes. The device/platform is more broadly applicable to any studies where circulation drives responses in multiple tissues (i.e. infection, metabolism, transport, etc.).

Thus, in various embodiments, an apparatus or device or platform according to the teachings herein may comprise, illustratively, a base and a lid wherein the base is comprised of a single solid cylinder containing four open-top chambers and can be rapidly 3D printed, wherein chambers decrease in diameter at their center (gradually, incrementally, or in a large step-change), creating a surface onto which culture substrates can be secured. By securing a membrane to this surface each basin is separated into two sub-chambers. Each of these sub-chambers is capable of harboring target cells, isolated from each other by the separating membrane. The base also incorporates two unique milli/micro-fluidics circuits which allow separate flow dynamics into the two sub-chambers. The first fluidics circuit allows flow to be directed across the lower sub-chamber, entering through a tube towards the bottom of the base, and exit through a second tube on towards the top of the base. The second fluidics allows flow to enter from the bottom of the base and exit the top. As such, multiple bases may be stacked or linked together in a manner providing fluid communication between stacked devices to provide increased model complexity.

It is noted that the various integrated fluidics circuits may be configured such that their respective fluid flows are primarily directed towards an incline such that the risk of bubble formation is greatly reduced. Finally, the base incorporates (illustratively) four recesses into the bottom, which line up with protrusion on the device lid (discussed below) thus ensuring a secure connection between two individual devices.

For example, assuming a plurality of devices configured as described herein with a base, lid, and a plurality of chambers formed therein, a first such device may have stacked upon it a second such device wherein the first and second devices may be in fluid communication with each other via one or more means. Similarly, the second such device may have stacked upon it a third device wherein the second and third devices may be in fluid communication with each other via one or more means, and wherein the first and third devices may also be in fluid communication with each other via one or more means. Such fluid communication means may comprise, for example, fluid communications between adjacent stacked devices via the second fluidics circuits of one or more chambers, via externally applied primary or first fluidics circuits from either common fluid sources or differing fluid sources, via the third fluidic circuit output port protruding from one device being coupled to an input port of another device, and/or some combination thereof.

The lid comprises a single solid cylinder (or other shape, depending on the base shape) containing, illustratively, four protrusions on top and bottom, and a port through which the second fluidics exit tube inserts. The bottom protrusions may be configured to be insertable into the upper portion of the base chambers; by doing so the volume of the upper sub-chamber is reduced allowing modeling of vascular circulation. The top protrusions will insert into recessions built into the bottom of the base, thus ensuring a secure connection between two individual devices. As mentioned above, the lid also contains a hole which must be positioned to allow the upper tube of the second internal fluidics circuit to pass through.

Various embodiments may be readily manufactured on a 3D printer, such as a Formlabs Form2 SLA 3D printing system, using the Formlabs Clear Resin or similar materials. It is noted that this resin consists of photoreactive methacrylated monomers and polymers which crosslink together under ultraviolet light. The various embodiments may be manufactured using any 3D printing technology capable of providing a rigid structure. Similarly the 3D printing device and technique can be altered such as by utilizing various curing principles.

Advantageously, the various embodiments find particular utility within the context of assessing physiological relevant tissue responses to nanomaterial exposures. The various embodiments may be implemented in a modular manner such they may be used to support a large number of tissue relevant investigations. For example, culturing relevant gut cells within a sub-chamber with fluid flow in the chamber opposite will enable directed investigations into metabolism and nutrient uptake, further complexed if one multiple devices are connected to mimic metabolic circuits (i.e. with hepatocytes). Overall, this device can be applied to fields including, but not limited to, infection, medicine, drug development/pharmaceuticals, cancer, biodefense and the like.

The various embodiments are well suited to numerous applications and provide an ability to harbor multiple single cultures (thereby increasing experimental sample size in a small footprint), while having a slightly larger physical size (thereby providing higher cell density and yield, and facilitating more and varied analysis techniques from less materials). The various embodiments are also capable of being linked together and supplied via a single nutrient medium (thereby more accurately representing human physiological circulation).

The various embodiments enable modelling of interactions of nanomaterials at secondary sites of exposure (e.g., inhalation to blood-brain-barrier) as well as chronic exposures to low-dose non-toxic nanomaterials. The various embodiments may be used as a platform for whole-body and targeted tissue investigations. Furthermore, the broad application of these embodiments enables use in non-exposure studies, such as for studying infections, metabolic aberrations, pharmaceuticals and the like. Other use cases include the study of pharmaceuticals (e.g., antimicrobials, personalized medicine, cancer treatments etc.), biodefense, and basic research.

Various embodiments advantageously provide a system that merges three dimensional printing, substrate composition, and milli/micro-fluidics to allow increased cell yield, physiological function, across both single and multi-organ platforms. The system enables bypassing the use of animals to determine single and multi-organ exposure events. There is great application of this device for Nanotoxicology as it allows in vitro assessment of nanomaterial translocation, biodispersion, accumulation, and clearance, which is crucial to understanding occupational exposures of nanomaterials.

It is noted that ability for cells to translocate nanomaterials from an apical to basal is a crucial cell behavior when assessing multiple tissue toxicity. The device is capable of creating multiple volumetric compartments within itself, with cells cultured at the interface between these compartments. Such a configuration, with respect to cell culture and orientation, we are able to simulate and transfer of nanomaterials from a basal to apical cell surface, furthermore we can model the shuttling of materials from circulation into tissue and vice versa.

It is noted that a materials' ability to be dispersed through the body is defined by how distal from the primary exposure site the material can reach. Many factors dictate this process, including local peptides in the circulation which contribute to a protein corona around the material, this corona recognized by tissues for uptake, and the material ability to stay suspended in the circulatory flow. This invention, when linked in series allows modeling of the hemodynamics between tissues, thus allowing us to determine how materials may travel between tissue segments. Furthermore, the invention can be expanded beyond the nanomaterial exposures to address other aspects of multi-tissue communication via circulation.

Accumulation and clearance of materials from organs and tissues is an important and crucial factor for determining toxicological responses. Indeed, the more readily an organ can clear potential toxins the less likely it is to suffer from the effects of said toxin. The invention, as designed, incorporates unique internal fluidics to allow us to sample in real-time the amount of nanomaterials which cross the endothelial barrier and enter tissue compartments. The ability to measure this activity in real-time enables us to determine kinetics of uptake, potential for accumulation and clearance.

Various modifications may be made to the systems, devices, platforms, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative system, device, platform, apparatus and/or method, and illustrative examples shown and described. Accordingly, departures may be made from such details without departing from the scope of the general inventive concept.

What is claimed is:

1. An apparatus, comprising:
a base, having a top surface and a bottom surface and having formed therein a plurality of open-top chambers and at least a first fluidics circuit;
each open top chamber being divided into respective upper and lower sub-chambers by a respective chamber separating membrane, wherein the upper and lower sub-chambers are defined by interior surfaces configured to allow organ culture substrates to be secured thereto;
the first fluidics circuit configured to support a first fluid flow between a first port on the base and second port on the base, the fluidics circuit being further configured to support a fluid flow through a lower sub-chamber of at least one of the plurality of open top chambers.

2. The apparatus of claim 1, further comprising:
a lid, having a top surface and a bottom surface, the bottom surface having formed thereon a plurality of protrusions, each protrusion configured to mate with a corresponding upper sub-chamber of the plurality of open top chambers.

3. The apparatus of claim 1, wherein the first fluidics circuit is further configured to support a fluid flow through a lower sub-chamber of at least a second one of the plurality of open top chambers.

4. The apparatus of claim 1, the base having formed therein a second fluidics circuit configured to support a second fluid flow between a bottom port on the base in fluid communication with a lower sub-chamber and a top port on the base in fluid communication with a respective upper sub-chamber.

5. The apparatus of claim 4, wherein each of the plurality of open top chambers has associated with it a respective second fluidics circuit for supporting a respective second fluid flow therethrough.

6. The apparatus of claim 4, the base having formed therein a third fluidics circuit configured to support a third fluid flow between a port on the base in fluid communication with an upper sub-chamber and a top port protruding through the top surface of the base convey thereby a portion of the first fluid flow.

7. The apparatus of claim 6, further comprising:
a lid, having a top surface and a bottom surface, the bottom surface having formed thereon a plurality of protrusions, each protrusion configured to mate with a corresponding upper sub-chamber of the plurality of open top chambers;
the lid having formed therein a recess configured to allow passage therethrough of a top port protruding through the top surface of the base.

8. The apparatus of claim 2, wherein the base and lid comprise 3D printed components.

9. The apparatus of claim 1, wherein the base comprises a cylindrical component having four chambers defined therein.

10. The apparatus of claim 1, wherein each chamber separating membrane is configured to isolate at least some of target cells harbored in the respective sub-chambers separated thereby.

11. The apparatus of claim 1, wherein interior chamber surfaces are configured to allow organ culture substrates to be secured thereto.

12. The apparatus of claim 11, wherein interior chamber surfaces are configured during a 3D printing process to allow organ culture substrates to be secured thereto using native 3D printing materials.

13. The apparatus of claim 11, wherein interior chamber surfaces are configured to allow organ culture substrates to be secured thereto using a treatment selected to promote adhesion and growth of the organ culture substrate.

14. The apparatus of claim 1, wherein at least one of the sub-chambers includes a growth matrix associated with a desired organ culture.

15. The apparatus of claim 1, wherein at least one of said lid protrusions is configured to extend into and substantially seal a corresponding upper sub-chamber.

16. The apparatus of claim 2, wherein said base comprises four open-top chambers and said lid comprises four corresponding protrusions.

17. The apparatus of claim 7, wherein said base comprises four open-top chambers and said lid comprises four corresponding protrusions.

18. The apparatus of claim 1, wherein the apparatus comprises a first apparatus and the top surface of the lid of the first apparatus is configured to cooperate with a bottom surface of a base of a second apparatus such that the first and second apparatus may be combined into a stacked apparatus.

19. The apparatus of claim 4, wherein:
the apparatus comprises a first apparatus and the top surface of the lid of the first apparatus is configured to cooperate with a bottom surface of a base of a second apparatus such that the first and second apparatus may be combined into a stacked apparatus;
wherein each upper sub-chamber of the first apparatus is in fluid communication with a respective lower sub-chamber of the second apparatus.

20. A system, comprising:
a base, having a top surface and a bottom surface and having formed therein a plurality of open-top chambers and at least a first fluidics circuit, each open top chamber being divided into respective upper and lower sub-chambers by a respective chamber separating membrane, wherein the upper and lower sub-chambers are defined by interior surfaces configured to allow organ culture substrates to be secured thereto, the first fluidics circuit configured to support a first fluid flow between a first port on the base and second port on the base, the fluidics circuit being further configured to support a fluid flow through a lower sub-chamber of at least one of the plurality of open top chambers; and
a lid, having a top surface and a bottom surface, the bottom surface having formed thereon a plurality of protrusions, each protrusion configured to mate with a corresponding upper sub-chamber of the plurality of open top chambers.
